(19) United States
(12) Patent Application Publication
(10) Pub. No.: US 2004/0236287 A1
(43) Pub. Date: Nov. 25, 2004

(54) Forward shielding medical needle device

(75) Inventors: Kirk D. Swenson, North Caldwell, NJ (US); Bradley Wilkinson, North Haledon, NJ (US); Volker Niermann, Bound Brook, NJ (US); Jamieson Crawford, New York, NY (US)

Correspondence Address: DAVID W. HIGHET, VP AND CHIEF IP COUNSEL, BECTON, DICKINSON AND COMPANY, 1 BECTON DRIVE, MC 110, FRANKLIN LAKES, NJ, 07417-1880, US

(73) Assignee: Becton Dickinson & Company

(21) Appl. No.: 10/444,537

(22) Filed: May 23, 2003

(51) Int. Cl. A61M 5/00

(52) U.S. Cl. 604/263; 604/177

(57) ABSTRACT

A shielding medical device includes a needle cannula, a hub, a release element, a needle shield, and a biasing member. The hub has a proximal end, a distal end, and a passageway in fluid communication with the needle cannula. The distal end of the hub supports the needle cannula. The release element is fixed to the hub. The needle shield is hollow and has a proximal end, a distal end, and a pair of wings extending laterally from opposing sides. The proximal end of the needle shield is removably secured to the release element. The needle shield is axially movable with respect to the needle cannula from a first position in which the needle cannula is exposed to a second position in which the needle cannula is shielded within the needle shield. The biasing member is used to move the needle shield from the first to second positions.

### BACKGROUND OF THE INVENTION

1. Field of the Invention

The present invention relates to a medical device for safe and convenient handling of used needle cannulas. More particularly, the present invention relates to a medical needle device, preferably winged needle device, that is adapted to automatically shield a needle cannula after it is used in a medical procedure.

2. Description of Related Art

Blood collection sets or intravenous (IV) infusion sets typically include a needle cannula having a proximal end, a distal end with a puncture tip, and a lumen extending therebetween. The proximal end of the needle cannula is mounted to a plastic hub having a central passage that communicates with the lumen in the needle cannula. A thin flexible thermoplastic tube is connected to the hub and communicates with the lumen in the needle cannula. The end of the plastic tube remote from the needle cannula may include a fixture for connecting the needle cannula to a separate medical device, such as a holder, a blood collection tube, and the like.

In order to reduce the risk of incurring an accidental needle-stick wound, protection of used needle cannulas becomes important. With concern about infection and transmission of diseases, methods and devices to cover used needle cannulas have become important and in great demand. For example, some needle assemblies commonly employ a safety shield that may be moved into shielding engagement with a used needle cannula without risking an accidental needle stick.

U.S. Pat. Nos. 5,085,639; 5,088,982; and 5,154,699 to Ryan disclose safety winged needle devices for use with blood collection sets or infusion sets. The safety needle devices of these patents include an inner tube and an outer tube having cooperating surfaces in contact with each other. The cooperating surfaces have mating grooves and ramps. By applying a force in opposing directions on the inner and outer tubes, the outer tube is moved forcibly along the ramps and into engagement with the grooves of the inner tube, thereby moving the outer tube, and therefore the outer shield, into a shielding position in place about the needle tip. However, the cooperating surfaces of such outer and inner tubes provide a frictional engagement that requires much force to overcome. Moreover, maintaining an appropriate grip on the safety needle device to forcibly move the outer and inner tubes apart to the fully shielded position can be difficult due to the profile of the safety needle device and the length of required travel to the shielded position.

U.S. Pat. No. 5,505,711 discloses a winged injector needle assembly including a needle cannula attached to a hub and a holder forming a shield portion. The hub is slidable within the shield portion. The needle assembly also includes a latching mechanism between the hub and the shield portion to maintain the hub in a forward position with the needle cannula extending from the shield portion. Upon grasping of and release of the latching mechanism, the shield portion may be moved forward to shield the needle cannula. U.S. Pat. No. 5,928,199 discloses a similar forward shielding needle assembly, which further includes a lock for locking the shield portion in the forward position shielding the needle cannula. Release of the latching mechanism is awkward due to the relation between the latching mechanism extending between the shield portion and the hub. Further, this device requires the operator to exert a substantial force to shield the needle cannula, and is prone to rotation of the needle cannula within the shield portion during the shielding operation.

U.S. Pat. Nos. 5,779,679 and 6,210,371 to Shaw disclose winged IV sets with a retracting needle assembly adapted to automatically retract a needle cannula within a safety shield upon release of a latch. The winged IV sets include a pair of latch wings associated with an outer shield that hold the needle assembly in an unretracted position, and which may be released to cause the needle assembly to be retracted within the outer shield. A spring drives the needle assembly rearward to the retracted position within the safety shield. However, the spring extends entirely around the needle assembly during and after retraction, which may cause complications during retraction. Also, since retraction of such a device is achieved by holding the safety shield, the user does not hold the needle cannula during retraction and therefore does not maintain control over the needle cannula. Thus, if the latch is released while the needle cannula is inserted in a patient, the spring force may cause an undesirable partial or full retraction of the needle cannula, instead of merely passively activating the device to allow for complete retraction upon removal from the patient.

In view of the foregoing, there is a continuing need for a shielding medical needle device adapted to shield a used needle cannula once a medical procedure is completed.

### SUMMARY OF THE INVENTION

The present invention relates generally to a shielding medical needle device for shielding used needle cannulas. The shielding medical needle device generally includes a needle cannula, a hub, a release element, a needle shield, and, preferably, a biasing member. The hub includes a proximal end, a distal end and a passageway extending therethrough in fluid communication with the needle cannula. The distal end of the hub supports the needle cannula. The release element is fixed to the hub. The needle shield is generally hollow and includes a proximal end, a distal end and a pair of wings extending laterally from opposing sides thereof. The proximal end of the needle shield is removably secured to the release element with at least a grippable or graspable portion of the hub extending out from the proximal end of the needle shield. The needle shield is axially movable with respect to the needle cannula from a first position in which the needle cannula is exposed to a second position in which the needle cannula is shielded within the needle shield. The biasing member extends between the distal end of the hub and the proximal end of the needle shield. In operation, activation of the release element releases the proximal end of the needle shield from securement, thereby causing the biasing element to axially urge the needle shield from the first position toward the second position.

The biasing member may be a spring. The outer surface of the hub and the inner surface of the needle shield may interact such that axial rotation of the hub with respect to the needle shield is inhibited during movement of the needle shield from the first position toward the second position. For example, the hub and the needle shield may have non-circular cross-sectional shapes, thereby inhibiting axial rotation of the hub with respect to the needle shield during movement of the needle shield from the first position toward the second position. The inner surface of the needle shield may also include splines for inhibiting the axial rotation of the hub with respect to the needle shield during movement of the needle shield from the first position toward the second position.

The medical device may include a flexible tube extending from the proximal end of the hub. The medical device may further include a locking element for securing the needle shield in the second position. The locking element may include a pair of flexible locking arms at the distal end of the hub. Each pair of locking arms may include a hook for releasable engagement with a corresponding detent in the proximal end of the needle shield.

The release element may include at least one flexible locking arm extending distally from the hub and include a hook for releasable engagement with a corresponding detent in the proximal end of the needle shield. The release element may further include a pair of flexible locking arms extending distally from the hub. Each pair of locking arms may include a hook for releasable engagement with a corresponding detent in the proximal end of the needle shield. The at least one flexible locking arm may extend distally from the hub to form a pocket between the flexible locking arm and an outer surface of the hub adjacent the distal end of the hub. The pocket may accommodate the biasing member. In such an embodiment, the medical device may further include a locking element including at least one flexible locking arm at the distal end of the hub having a hook for releasable engagement with the detent in the proximal end of the needle shield when the needle shield is in the second position.

The release element may alternatively comprise a rotational locking engagement between the hub and the proximal end of the needle shield. A rotatable collar may extend from the hub. The rotatable collar may be independently rotatable with respect to the hub. The rotatable collar may be in releasable engagement with the proximal end of the needle shield when the needle shield is in the first position. The rotational locking engagement may be a threaded engagement.

The release element may also comprise at least one pivot arm extending laterally along the hub. The pivot arm may be in releasable engagement with the proximal end of the needle shield. The release element may further include a pair of pivot arms extending laterally along opposing outer sides of the hub. The pair of pivot arms may be in releasable engagement with corresponding surfaces on the proximal end of the needle shield.

In yet another embodiment, the medical device of the present invention includes a needle cannula, a hub, a needle shield, and a biasing member. The hub includes a proximal end, a distal end and a passageway extending therethrough in fluid communication with the needle cannula. The distal end of the hub supports the needle cannula. The needle shield is generally hollow and includes a proximal end, a distal end and a pair of wings extending laterally from opposing sides thereof. The needle shield is adapted to encompass the hub with at least a portion of the hub extending from the proximal end of the needle shield. The needle shield is axially movable with respect to the needle cannula from a first position in which the needle cannula is exposed to a second position in which the needle cannula is shielded within the needle shield. The biasing member extends between the hub and the needle shield and exerts a biasing force to urge the needle shield distally toward the second position. In operation, when the needle shield is in the first position the shield and the hub exhibit a mutual friction greater than the biasing force exerted by the biasing member when the needle shield is in the first position.

The mutual friction between the shield and the hub may be in the range of about 120% to about 1000% of the biasing force exerted by the biasing element. Desirably, the mutual friction is provided through surface texture on at least one of an outside surface of the hub or an inside surface of the needle shield. The portion of the hub that extends from the proximal end of the needle shield may include a surface grip and the needle shield may include a surface grip, such that opposing forces applied against the surface grip of the hub and the surface grip of the needle shield releases the mutual friction between the hub and the shield and the biasing element causes axial movement of the needle shield toward the second position.

The medical device may further include a locking element for securing the needle shield in the second position. An inner surface of the needle shield may include a first surface feature at the proximal end thereof and a second surface feature at the distal end thereof. The external surface of the hub may include a surface feature for interference engagement with the first surface feature of the needle shield when the needle shield is in the first position for retaining the needle shield in the first position, and for interference engagement with the second surface feature of the needle shield when the needle shield is the second position for securing the needle shield in the second position. The first and second surface features of the needle shield may comprise first and second detents, respectively, and the surface feature of the hub may comprise a bump for respective interference engagement with the first and second detents of the needle shield.

Further details and advantages of the present invention will become apparent from the following detailed description when read in conjunction with the drawings, wherein like parts are designated with like reference numerals and lower case letters are included where necessary to identify specific embodiments of the invention.

### DESCRIPTION OF THE DRAWINGS

FIG. 1 is a longitudinal cross sectional view of a medical device in accordance with the present invention showing a needle shield of the medical device locked in a first, retracted position proximate to a needle hub of the medical device;

FIG. 2 is a longitudinal cross sectional view of the medical device of FIG. 1 showing the needle shield in a second, extended position;

FIG. 3A is a transverse cross sectional view of the medical device of FIG. 1 showing the interaction between the needle hub and needle shield according to one embodiment of the present invention;

FIG. 3B is a transverse cross sectional view of the medical device of FIG. 1 showing the interaction between the needle hub and needle shield according to another embodiment of the present invention;

FIG. 4 is a longitudinal cross sectional view of another embodiment of the medical device of the present invention including a rotatable collar for locking the needle shield in the first position;

FIG. 5 is a longitudinal cross sectional view of the medical device of FIG. 4 showing the needle shield in the second, extended position;

FIG. 6 is a longitudinal cross sectional view of yet another embodiment of the medical device of the present invention including pivot arms on the needle hub for locking the needle shield in the first position;

FIG. 7 is a longitudinal cross sectional view of the medical device of FIG. 6 showing the needle shield in the second, extended position;

FIG. 8 is a longitudinal cross sectional view of the medical device according to a still further embodiment of the present invention, wherein the needle shield is locked in the first position by frictional interference between the needle hub and the needle shield;

FIG. 9 is a longitudinal cross sectional view of the medical device of FIG. 8 showing the needle shield locked in the second position by frictional interference between the needle hub and needle shield; and

FIG. 10 is a perspective view of the medical device of the present invention incorporated as part of a blood collection set.

### DETAILED DESCRIPTION

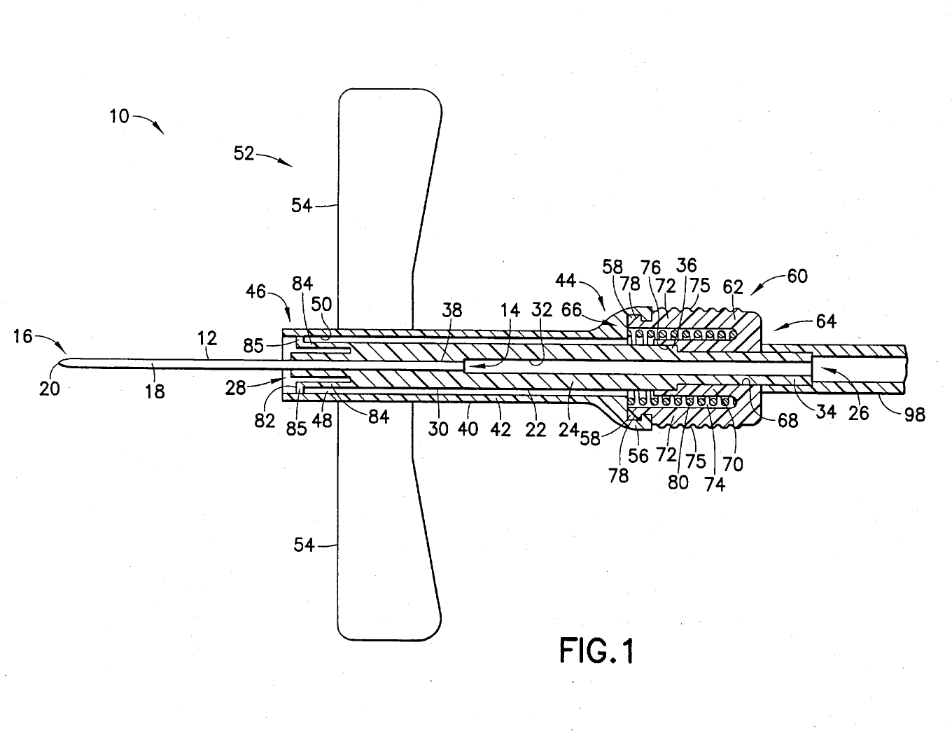

FIGS. 1-10 generally illustrate several embodiments of a medical device 10 according to the present invention. The several embodiments of the medical device 10 may be used as part of a fluid collection set used to collect blood or other fluids from the body of a human or animal. The medical device 10 of the present invention is generally a shieldable device adapted to enclose or surround a used needle cannula at the end of a fluid collection procedure. Preferably, the medical device 10 includes a portion that is automatically biased to a safety, needle-enclosing position upon activation by a user of the medical device 10, as discussed in detail hereinafter.

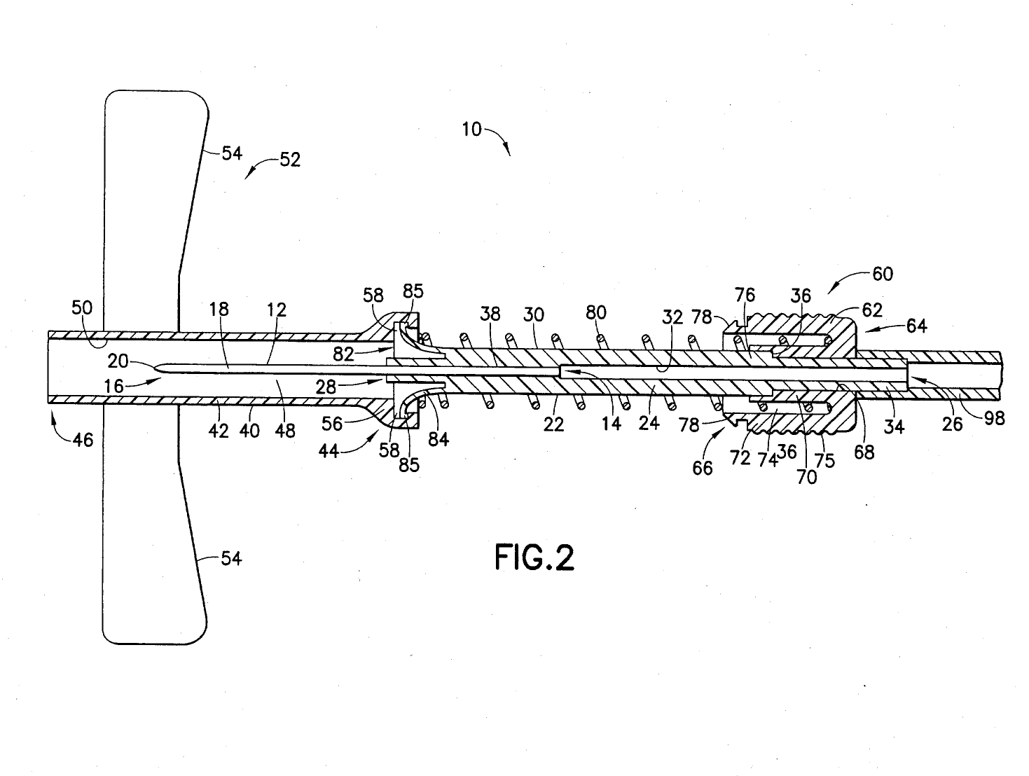

Referring to FIGS. 1 and 2, a first embodiment of the medical device 10 is shown. The medical device 10 generally includes a needle cannula 12, a hub 22, a needle shield 40, a release element 60 releasably connecting the hub 22 and needle shield 40 and a biasing member 80, for actuating (i.e., moving) the needle shield 40 to the safety, needle-enclosing position or configuration. The hub 22 is adapted for connection to a receptacle (not shown) of, for example, a blood collection set by way of a flexible tube 98 by means and procedures known in the art.

The needle cannula 12 includes a proximal end 14 and distal end 16 and defines a lumen 18 extending from the proximal end 14 to the distal end 16. The distal end 16 of the needle cannula 12 is beveled to define a sharp puncture tip 20 suitable for venipuncture into the blood vessel of a human or animal. The puncture tip 20 is preferably designed to provide ease of insertion and minimal discomfort during venipuncture. A removable protective cover (not shown) may be positioned over the distal end 16 of the needle cannula 12 to prevent accidental needle-stick wounds prior to using the medical device 10 in a medical procedure and to protect the puncture tip 20 prior to use.

The hub 22 is preferably a unitary structure, which is desirably molded from a thermoplastic material. The hub 22 has a generally tubular-shaped body 24 with a proximal end 26 and a distal end 28. The body 24 has an outer surface 30 and defines an internal passageway or lumen 32 extending from proximal end 26 to distal end 28. The passageway 32 communicates with the lumen 18 defined in the needle cannula 12 to enable fluid, such as blood, to pass through the medical device 10 and to the tube 98 connecting the medical device 10 to the blood collection receptacle.

The proximal end 14 of needle cannula 12 is received within and supported by the distal end 28 of the body 24. Particularly, the proximal end 14 of the needle cannula 12 is disposed in the passageway 32, with the distal end 16 of needle cannula 12 projecting outward from the distal end 28 of the body 24. The needle cannula 12 is preferably secured to the hub 22 through the use of an appropriate medical grade adhesive, mechanical means, or the like. In particular, the proximal end 14 of the needle cannula 12 may be secured adhesively within the passageway 32 at the distal end 28 of the body 24 of the hub 22. The proximal end 26 of the body 24 may include a tapered or reduced diameter portion 34 having a shoulder 36, whose function will be discussed hereinafter. The proximal end 26 of the body 24 is generally adapted to cooperate with the tube 98 used to connect the medical device 10 to, for example, a blood collection receptacle. The passageway 32 at the distal end 28 of the body 24 preferably includes a reduced diameter portion or area 38 sized to accept and support the proximal end 14 of the needle cannula 12.

The needle shield 40 includes a generally tubular body or housing 42 adapted to generally coaxially receive the hub 22, as shown in FIG. 1. The needle shield 40 is preferably a unitary structure, which is desirably molded from a thermoplastic material. The body 42 of the needle shield 40 includes a proximal end 44 and a distal end 46. The body 42 defines a central bore or passageway 48 extending between the proximal end 44 and distal end 46. The passageway 48 is sized to coaxially receive the body 24 of the hub 22. Preferably, an inner wall or surface 50 forming the passageway 48 substantially slidably cooperates with the outer surface 30 of the body 24 of the hub 22, with only a small gap therebetween.

The needle shield 40 includes a butterfly-type wing assembly 52 including a pair of wings 54 extending laterally from the body 42 of the needle shield 40. The pair of wings 54 extends from opposing sides of the body 42 for stabilizing the medical device 10 in contact with the body of a patient during a blood collection or other fluid collection procedure. In particular, the wings 54 assist in positioning and placing the medical device 10 and associated blood collection set during a blood collection procedure, for example. The wings 54 are adapted to lie flat against the surface of a patient's skin. The wings 54 may be constructed of a flexible material such that at least one, and preferably both, of the wings 54 may be bent toward each other and brought together between the fingers of the user to assist in positioning and placing the medical device 10 during venipuncture. The proximal end 44 of the body 42 of the needle shield 40 includes an outwardly bulged or tapered portion 56, which defines one or more recesses or detents 58 for coacting with portions of the hub 22, as discussed further hereinafter.

The needle shield 40 is generally movable between a first or retracted position coaxially surrounding the hub 22 and a second, extended or needle-shielding position, wherein the needle cannula 12 is enclosed or disposed within the needle shield 40 fully covering the needle cannula 12 and, in particular, the puncture tip 20. The first or retracted position of the needle shield 40 is shown in FIG. 1 and the second or extended position of the needle shield 40 is shown in FIG. 2. In the first position of the needle shield 40, the distal end 28 of the hub 22 preferably does not extend (i.e., project) beyond the distal end 46 of needle shield 40. However, the distal end 16 of the needle cannula 12 projects outward from the needle shield 40 for use in a fluid collection procedure. Moreover, the inner surface 50 of the needle shield 40 is in substantial cooperating (i.e., slidable) engagement with the outer surface 30 of the hub 22, as indicated previously.

The needle shield 40 is secured releasably in the first position by a release element 60. The release element 60 is preferably fixed to the hub 22, for example, by a medical grade adhesive or by mechanical methods. The release element 60 has a body 62 with a first or proximal end 64 and second or distal end 66. The body 62 defines an internal bore or passageway 68 adapted to receive the hub 22. The body 62 of the release element 60 is generally comprised of an annular-shaped inner member 70 defining the passageway 68, and one or more locking arms 72 located about the inner member 70. An annular groove or pocket 74 is formed or defined by the inner member 70, the locking arms 72, and the outer surface 30 of the hub 22. The function of the annular groove or pocket 74 will be described hereinafter. While illustrated as a separate structure from the hub 22, the release element 60 may be integrally formed as part of the hub 22. The release element 60 is desirably molded from a thermoplastic material, either separate from or integrally with the hub 22 as indicated. Thus, the release element 60 may be considered to be part of the hub 22 in accordance with the present invention and generally forms a “grippable” or “graspable” portion of the hub 22, which extends proximally out from the needle shield 40. The release element 60 thus forms a convenient handle means for manipulating the medical device 10. An outer surface 75 of the body 62 of the release element 60 and, more particularly, the locking arms 72 may be textured providing a surface grip for ease in grasping and manipulating the medical device 10 during a fluid collection procedure. When formed as a separate structure, the release element 60 in this embodiment of the medical device 10 is preferably fixedly connected to the hub 22, for example by a medical grade adhesive or by mechanical methods, so that the locking arms 72 are positioned at fixed locations around the hub 22.

The body 62 of the release element 60 generally coaxially receives the proximal end 26 of the body 24 of the hub 22, as illustrated in FIGS. 1 and 2. In particular, the reduced diameter portion 34 at the proximal end 26 of the body 24 of the hub 22 is received in the annular-shaped inner member 70, and preferably extends proximally outward from the proximal end 64 of the body 62 of the release element 60 to cooperate with the tube 98. The inner member 70 defines a distal recess 76 opening to the internal passageway 68. The distal recess 76 has a larger diameter than the diameter of the internal passageway 68. The distal recess 76 is adapted to cooperate with the reduced diameter or tapered portion 34 defined by the body 24 of the hub 22. The shoulder 36 of the reduced diameter portion 34 engages the distal recess 76 in the first position of the needle shield 40 and prevents axial movement of the release element 60 along the body 24 of the hub 22 in the distal direction, as is most apparent in FIG. 2.

The locking arms 72 extend distally along the inner member 70 and terminate in integrally formed barbs or hooks 78 adapted to mate with the detents 58 provided at the proximal end 44 of the needle shield 40. In particular, as indicated previously, the detents 58 are formed in the outwardly bulged portion 56 at the proximal end 44 of the body 42 of the needle shield 40. The detents 58 and locking arms 72 form a locking element of the medical device 10. The engagement of the hooks 78 with the detents 58 secures the needle shield 40 in the first or retracted position. As indicated previously, the release element 60 is preferably fixedly connected to or integrally formed with the hub 22. Thus, the locking arms 72 and, more particularly, the hooks 78 are positioned at fixed locations around the hub 22 opposite the detents 58 to properly engage the detents 58 and secure the needle shield 40 in the first or retracted position. It is preferred that the medical device 10 be preset by the manufacturer with the needle shield 40 in the first, shield-retracted position with a cover enclosing the puncture tip 20. The user of the medical device 10 then only needs to make the appropriate connection to, for example, a blood collection receptacle, and remove the needle cover to make the medical device 10 ready for use.

As stated, the body 62 of the release element 60 is molded of a thermoplastic material, thereby making the locking arms 72 inherently flexible. The flexible locking arms 72 permit the locking arms 72 to be compress inward toward the hub 22, which releases the hooks 78 of engagement with the detents 58 and enables the needle shield 40 to be moved to the second, needle-shielding position. The present invention envisions that once the locking arms 72 are released of engagement with the needle shield 40, the needle shield 40 may be moved by the user of the medical device 10 to the second position or moved automatically to the second, needle-shielding position by means discussed hereinafter. It will be apparent that the location for the locking arms 72 and corresponding detents 58 may be reversed in accordance with the present invention. Accordingly, the locking arms 72 may be provided at the proximal end 26 of the body 24 of the hub 22 and extend proximally toward the release element 60. The detents 58, previously located on the hub 22, may be provided at the distal end 66 of the body 62 of the release element 60, such as defined in an annular outer member located coaxially out from and connected to the inner member 70.

The medical device 10 preferably further includes a biasing member 80 adapted to act between the release element 60 formed with or connected to the hub 22 and the needle shield 40 to automatically (i.e., passively) move the needle shield 40 to the second, needle-shielding position. The biasing member 80 is preferably a compression spring or the like. The biasing member 80 is disposed in the pocket 74 defined by the inner member 60, the locking arms 72 and outer surface 30 of the hub 22, as shown in FIG. 1. The biasing member 80 is maintained in a compressed state by engagement of the hooks 78 of the locking arms 72 with the detents 58 in the needle shield 40. The biasing member 80 is retained in a compressed state in the pocket 74 by contact or engagement with the proximal end 44 of the body 42 of the needle shield 40. The distal end of the biasing member 80 may be secured to the proximal end 44 of the body 42 of the needle shield 40 by a suitable medical grade adhesive known in the art or by a fixed mechanical connection. Likewise, the proximal end of the biasing member 80 may be adhesively or mechanically secured in the pocket 74. Once the biasing member 80 is released by disengaging the hooks 78 on the locking arms 72 from the corresponding detents 58, the biasing member 80 exerts an axial expansion force against the needle shield 40, and in particular, against the proximal end 44 of the needle shield 40, relative to the hub 22 and release element 60. The needle shield 40 is urged by the biasing member 80 to the second, needle-shielding position, wherein the needle shield 40 covers the needle cannula 12 (i.e., FIG. 2). The biasing member 80 also substantially prevents the needle shield 40 from moving proximally back toward the release element 60 and proximal end 44 of the body 42 of the needle shield 40 by providing a counter-acting biasing force against such a movement.

The medical device 10 further includes a locking element 82 provided at the distal end 28 of the body 24 of the hub 22. In particular, the locking element 82 is preferably integrally formed as part of the distal end 28 of the body 24. The locking element 82 preferably comprises two outward-biased locking arms 84 each having a barb or hook 85 adapted to cooperate with the detents 58 defined in the body 42 of the needle shield 40, at the proximal end 44 of the needle shield 40. The outward-biased locking arms 84 are restrained in the passageway 48 of the needle shield 40 when the needle shield 40 surrounds the hub 22 by contact with the inner surface 50 of the needle shield 40. Once the biasing element 80 is released by the release element 60 and the needle shield 40 has begun moving to the second, needle-shielding position, the locking arms 84 slide along in contact with the inner surface 50 of the needle shield 40 until the hooks 85 reach the detents 58 formed in the needle shield 40. The outward-biased locking elements 82 and, more particularly, the hooks 85 then snap into engagement with the detents 58 and prevent the needle shield 40 from moving proximally along the hub 22 toward the release element 60, thereby preventing re-exposure of the needle cannula 12.

Referring to FIGS. 1, 2, 3A, and 3B, the outer surface 30 of the hub 22 and the inner surface 50 of the needle shield 40 preferably interact such that any axial rotation of the hub 22 with respect to the needle shield 40 is inhibited during the sliding, expanding movement of the needle shield 40 from the first position to the second position. This ensures that the locking barbs or hooks 85 of the locking arms 84 mate properly with the detents 58 when the needle shield 40 is moved from the first to the second positions.

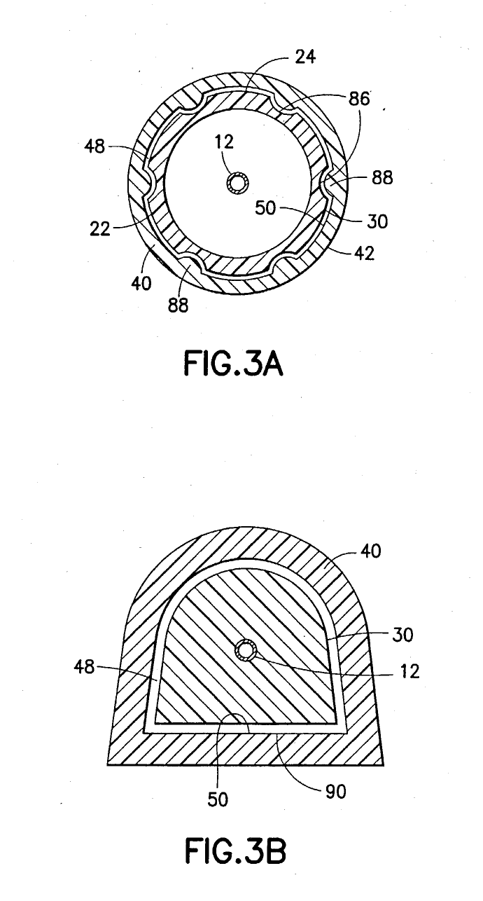

In one embodiment, the hub 22 and needle shield 40 may each have a substantially circular cross section as shown in FIG. 3A. To prevent rotation of the needle shield 40 relative to the hub 22, the hub 22 defines at least one and, preferably, a plurality of spline grooves or detents 86 in the outer surface 30 of the body 24 of the hub 22. The spline grooves or detents 86 mate with one or more splines 88 formed on the inner surface 50 of the body 42 of the shield member 40. The interaction of the splines 88 and spline detents 86 prevents rotation of the needle shield 40 relative to the hub 22, when the needle shield 40 is extended from the first to the second, needle-shielding position. The mating splines 88 and spline detents 86 may be replaced by any suitably equivalent surface feature on the outer surface 30 of the hub 22 and the inner surface 50 of the needle shield 40, which prevents relative rotation between these structures when the needle shield 40 is extended to the second, needle-shielding position. Additionally, the locations of the splines 88 and spline detents 86 (i.e., surface features) may be reversed in accordance with the present invention.

In an alternative embodiment shown in FIG. 3B, the hub 22 and needle shield 40 may each have a non-circular cross section, for example, with one planar side 90. As will be appreciated by those skilled in the art the non-circular cross sections of the hub 22 and needle shield 40 will prevent relative rotation between these elements. Any polygonal shaped structure would be suitable for the shape of the hub 22 and needle shield 40 in accordance with the embodiment of the medical device 10 shown in FIG. 3B.

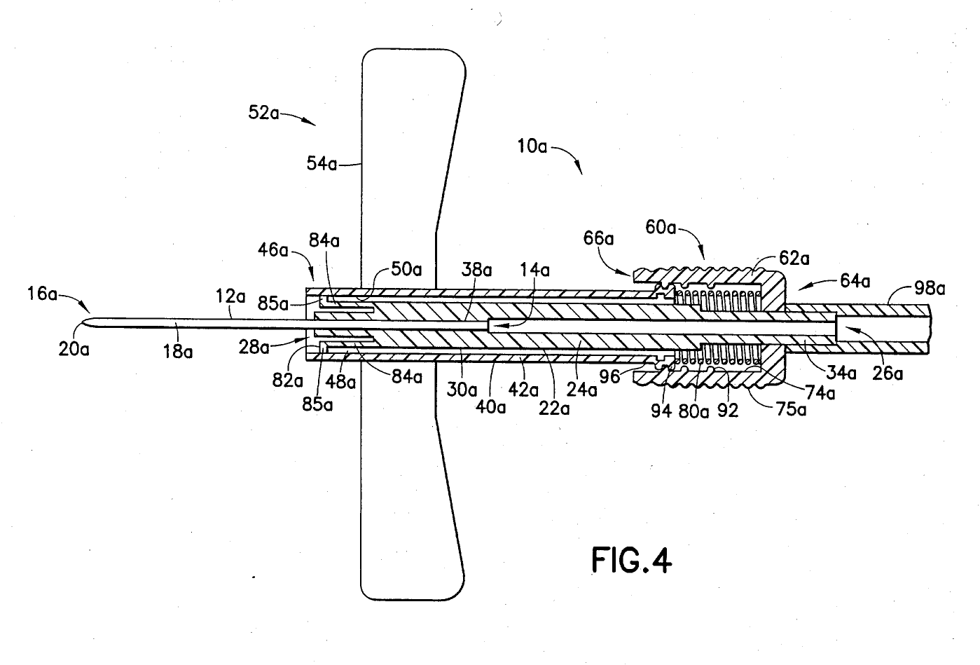

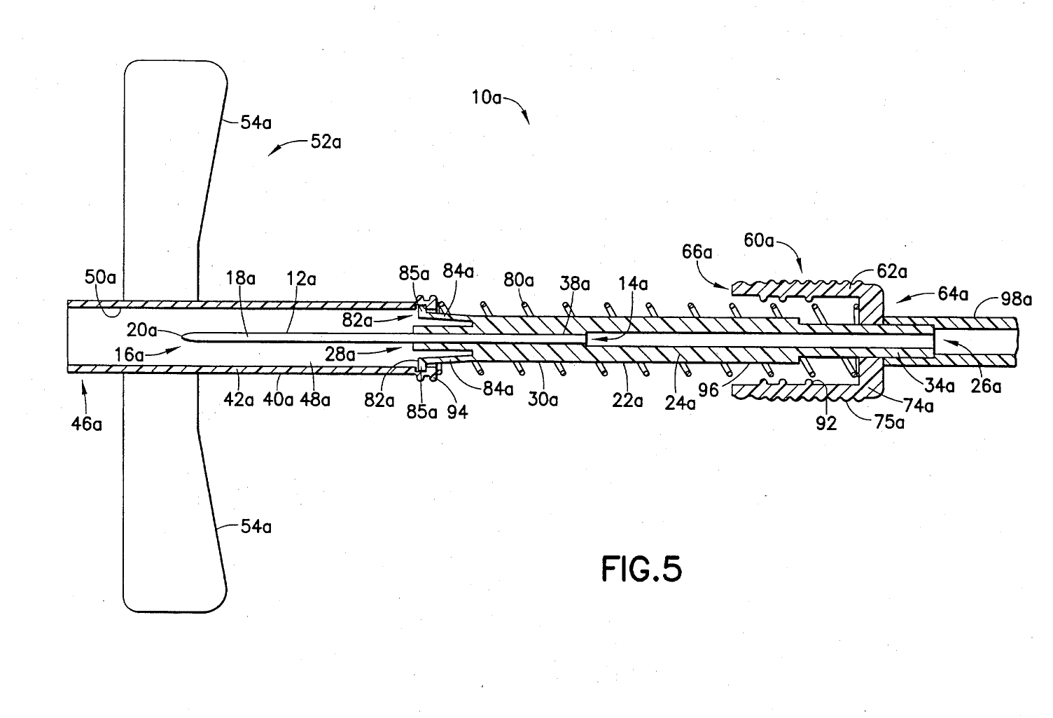

Another embodiment of the medical device 10a is shown in FIGS. 4 and 5. The medical device 10a of FIGS. 4 and 5 is substantially similar to the embodiment of the medical device 10 shown in FIGS. 1 and 2. However, the locking engagement between the release element 60a and needle shield 40a is slightly modified. The release element 60a in this embodiment is provided as a rotatable locking collar that is received on the proximal end 26a of the body 24a of the hub 22a. In particular, the rotatable release element 60a is received about the reduced diameter portion 34a at the proximal end 26a of the body 24a of the hub 22a. The release element (i.e., rotatable locking collar) 60a includes internal threads 92 adapted to cooperate with external threads 94 formed on an outer surface 96 of the body 42a of the needle shield 40a. The external threads 94 are provided at the proximal end 44a of the body 42a of the needle shield 40a. The interaction between the internal and external threads 92, 94, as shown in FIG. 4, prevents the needle shield 40a from extending to the second, needle-shielding position. The needle shield 40a is released to extend to the second, needle-shielding position by rotating the release element 60a to unthread the release element 60a from the proximal end 44a of the needle shield 40a. Once the release element 60a is completely unthreaded from the needle shield 40a, the biasing member 80a automatically moves the needle shield 40a to the second, needle-shielding position. The biasing member 80a may be omitted and the user of the medical device 10a may provided the necessary axial force to move the needle shield 40a to the second, needle-shielding position. As will be apparent, the threaded locking arrangement between the release element 60a and the needle shield 40a may have a standard clockwise-to-thread, counter-clockwise-to-unthread configuration, or the opposite configuration. Rotation of the release element 60a to unthread the release element 60a from the needle shield 40a will cause the needle shield 40 to begin to move distally toward the needle cannula 12 until engagement is released completely whereupon the biasing member 80a moves the needle shield 40a to the second, needle-shielding position automatically. The needle shield 40a is locked in the second, needle-shielding position in the same manner as discussed previously in connection with the medical device 10 of FIGS. 1 and 2.

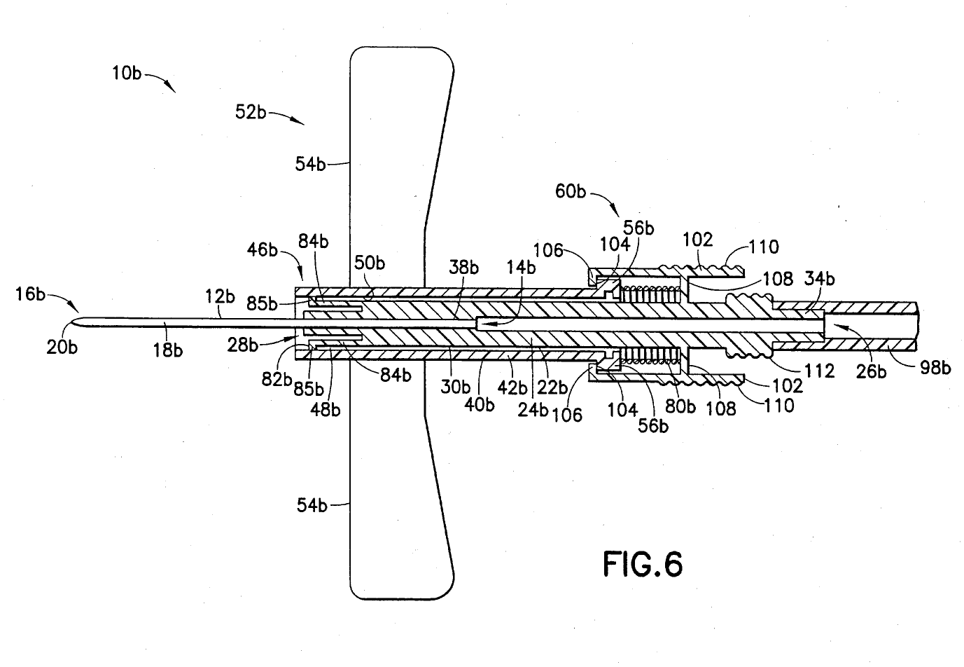

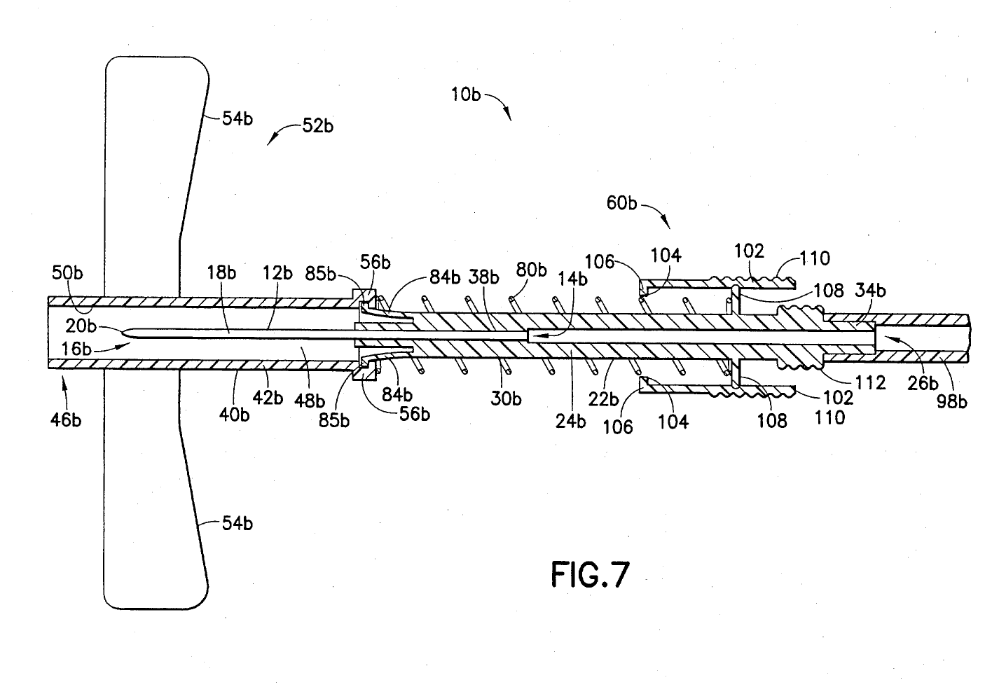

A further embodiment of the medical device 10b of the present invention is shown in FIGS. 6 and 7. The medical device 10b of FIGS. 6 and 7 is substantially similar to the embodiments of the medical device 10 shown in FIGS. 1, 2 and 4, 5. However, the locking engagement between the release element 60b and needle shield 40b is again slightly modified. The locking arms 72b of the release element 60b are now provided as pivot arms 102 adapted to mate with corresponding engagement surfaces 104 of the body 42b of the needle shield 40. The pivot arms 102 are pivotally connected to the body 24b of the hub 22b, at the proximal end 26b of the body 24b. The pivot arms 102 include hook portions or members 106 adapted to releasably mate with the corresponding engagement surfaces 104. The engagement surfaces 104 are formed by the bulged portion 56b of the body 42b of the needle shield 40b, as shown in FIG. 6. The bulged portion 56b preferably now has a rectangular shape to form the engagement surfaces 104. The interaction between the hook portions 106 of the pivot arms 102 and the engagement surfaces 104 at the proximal end 44b of the needle shield 40b prevents the needle shield 40b from extending to the second, needle-shielding position. The needle shield 40b is released to extend to the second, needle-shielding position by rotating or pivoting the pivot arms 102 about their respective pivot axes 108, which may be, for example, hinges or fulcroms, which releases the hook portions 106 of engagement with the engagement surfaces 104. The pivot arms 102 preferably include textured actuating members or tabs 110 for pivoting the pivot arms 102 and releasing their engagement with the engagement surfaces 104. The texturing is provided to minimize the possibility that the user's fingers will slip from the pivot arms 102 when attempting to release the needle shield 40b. Once the release element 60b (i.e., pivot arms 102 and engagement surfaces 104) is fully released, the biasing member 80b automatically moves the needle shield 40b to the second, needle-shielding position. The body 24b of the hub 22b may define a stop member 112 for limiting the axial distance that the tube 98 may extend along the proximal end 26b of the body 24b of the hub 22b. The needle shield 40b is locked in the second, needle-shielding position in the same manner as discussed previously in connection with the medical devices 10, 10a of FIGS. 1, 2 and 4, 5, respectively.

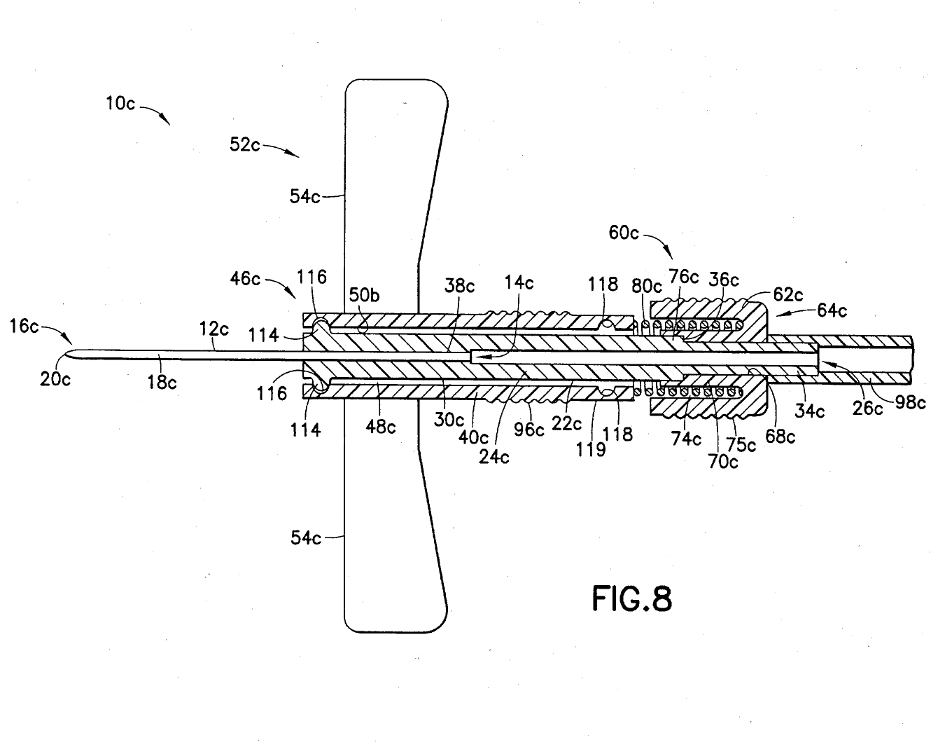

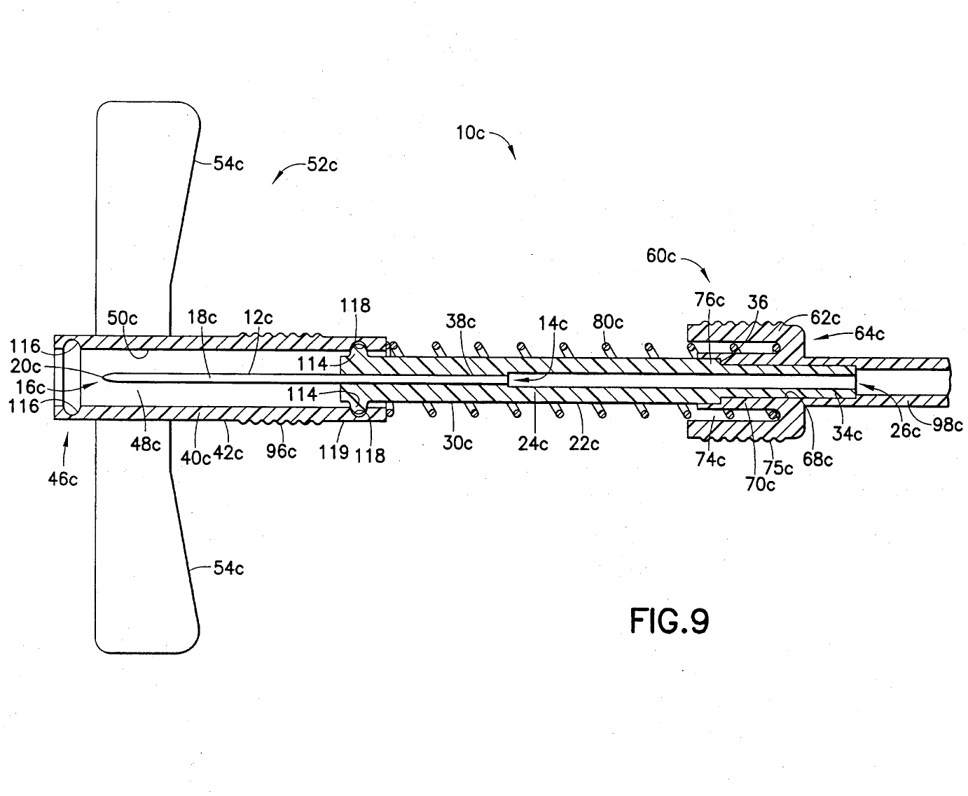

A final embodiment of the medical device 10c is shown in FIGS. 8 and 9. The medical device 10c of FIGS. 8 and 9 is substantially similar to the embodiments of the medical device 10, 10a, 10b shown in FIGS. 1, 2 and 4, 5 and 6, 7. However, the locking engagement between the release element 60c and needle shield 40c is replaced by a direct locking connection between the needle shield 40c and hub 22c. The release element 60c may be provided as a rotatable collar in a similar manner to the medical device 10a of FIGS. 4 and 5, or as a fixed and “grippable” element as discussed in connection with FIGS. 1 and 2. The release element 60c houses the biasing member 80 in the manner discussed previously (i.e., in pocket 74c).

The locking arms 84 of the locking element 82 provided at the distal end 28 of the hub 22 of the medical device 10 of FIGS. 1 and 2 are replaced in this embodiment with one or more raised members 114, for example one or more splines or bumps. Alternatively, the raised members 114 may comprise a single raised member extending circumferentially around the distal end 28c of the hub 22c. The raised members 114 are preferably integrally formed as part of the body 24c of the hub 22c, or provided as an external structure added to the distal end 28c of the body 24c. The raised members 114 are adapted to cooperate with one or more corresponding detents or recesses 116 defined in the inner surface 50c of the needle shield 40c substantially at the distal end 46 of the needle shield 40, and one or more corresponding detents or recesses 118 defined in the inner surface 50c of the needle shield 40c substantially at the proximal end 44 of the needle shield 40. Alternatively, the detents or recesses 116, 118 may extend circumferentially around the inner surface 50c of the needle shield 40c (i.e., as a groove) when the raised members 114 are formed in a corresponding manner.

The interaction between the raised members 114, or surface features or textures, and the corresponding detents 116, 118, or surface features or textures, maintains the needle shield 40 in the first, retracted position or second, extended position. In particular, the raised members 114 will be received in the distal detents 116 when the needle shield 40c is in the first or retracted position, and will be received in the proximal detents 118 when the needle shield 40c is in the second or extended position. The frictional engagement of the raised member 114 and the distal detents 116 (i.e., corresponding or interacting surface features or textures) is preferably sufficient to prevent the biasing member 80c from moving the needle shield 40c to the second, needle-shielding position. In particular, the mutual friction between the outer surface 30c of the hub 22c (i.e., raised members 114) and inner surface 50 of the needle shield 40c (i.e., distal detents 116) is greater than the biasing force of the biasing member 80, which maintains the needle shield 40c in the first position. For example, the mutual friction between the outer surface 30c of the hub 22c and inner surface 50c of the needle shield 40c may be about 120-1000% of the biasing force of the biasing member 80c.

When the needle shield 40c is to be extended to the second, needle-shielding position, the user of the medical device 10c must urge or move the needle shield 40c in a distal direction, which dislodges the raised members 114 from the distal detents 116. The dislocation of the raised members 114 from the distal detents 116 reduces the frictional engagement between the hub 22c and needle shield 40c, which allows the biasing force exerted by the biasing member 80c to move the needle shield 40c automatically (i.e., passively) to the second, needle-shielding position. The biasing member 80c interacts with the proximal end 26c of the hub 22c in the manner discussed previously to move the needle shield 40 to the needle-shielding position.

Once the biasing force of the biasing member 80c is released, the needle shield 40c moves distally to cover the needle cannula 12c. The raised members 114 seat in the proximal detents 118 defined in the inner surface 50c of needle shield 40c at the proximal end 44c of the needle shield 40c. The frictional interaction between the outer surface 30c of the hub 22c (i.e., raised members 114) and the inner surface 50c of the needle shield 40c (i.e., proximal detents 118) and the biasing force of the biasing member 80c prevents the needle shield 40c from moving proximally and uncovering the needle cannula 12c. Thus, an interference engagement between the hub 22c and needle shield 40c is used to prevent relative movement between these elements until the user of the medical device 10c moves the needle shield 40c distally to disengage the raised members 114 from the distal detents 116, and further prevents the needle cannula 12c from re-emerging from the needle shield 40c once the needle shield 40c is moved to the second position. The outer surface 96c of the body 42c of the needle shield 40c may comprise a textured surface 119 forming a surface grip for grasping by the user of the medical device 10c. In particular, in operation, the user of the medical device 10c described hereinabove will apply opposing axial forces between the textured surface 119 of the needle shield 40c and the textured surface 75c of the release element 60c (or hub 22c) to overcome the mutual friction between the outer surface 30c of the hub 22c and the inner surface 50c of the needle shield 40c and release the biasing member 80c, which then moves the needle shield 40c to the second position automatically. The raised members 114 and corresponding distal and proximal detents 116, 118 may be replaced by any suitable mating or corresponding structures, surface features, or surface textures that exhibit sufficient mutual friction to overcome the biasing force of the biasing member 80c.

As indicated previously, the medical device 10 of the present invention is preferably packaged with the needle shield 40 in the first position, as shown, for example, in FIGS. 1, 4, 6 and 8. Prior to use, the medical device 10 is removed from its package (not shown) for attachment with the flexible tube 98, which is attached to an appropriate fluid collection receptacle. To prepare the medical device 10 for use with, for example, a blood collection set and other like devices, the user grasps the medical device 10 and removes the protective cover (not shown) to expose the puncture tip 20 of needle cannula 12. The user then inserts the puncture tip 20 into a targeted blood vessel of a patient. During such positioning, at least one of the wings 54 may be bent inwardly toward the other by the user's fingers to facilitate positioning and placing the medical device 10. Upon completion of the procedure, such as when all desired samples have been drawn, the needle cannula 12 is withdrawn from the patient. After removal of the needle cannula 12 from the patient, activation of the safety feature of the medical device 10 allows the compressed biasing member 80 to expand thereby urging the needle shield 40 to lock in the second, needle-shielding position in the manner discussed previously in connection with the various embodiments of the medical device 10.

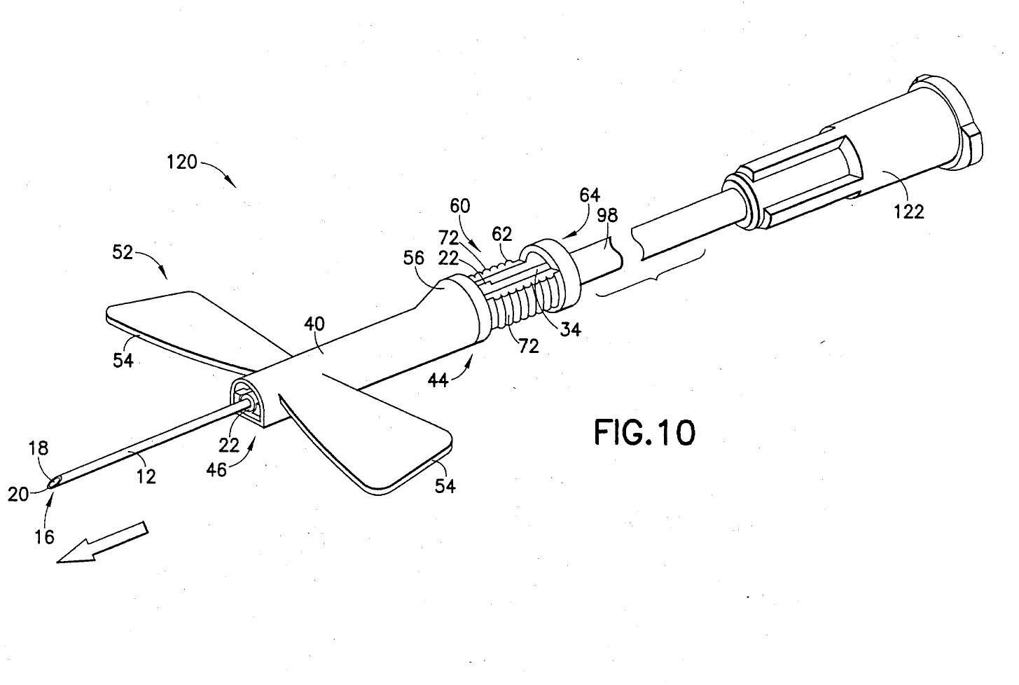

Referring to FIG. 10, the medical device of the present invention may be incorporated as part of a blood collection assembly 120, as indicated previously. The tube 98 is connected to the proximal end 26 of the hub 22 is further connected to a blood collection receptacle 122. The blood collection receptacle 122 is adapted to receive a fluid collection tub (not shown) such as a blood collection tube.

While the medical device of the present invention was described in terms of several preferred embodiments for use in connection with bodily fluid collection systems and like devices, the present invention may take many different forms. The preferred embodiments shown in the drawings and described hereinabove in detail are to be considered as exemplary of the principles of the invention and are not intended to limit the invention to the embodiments illustrated. Various other embodiments will be apparent to and readily made by those skilled in the art without departing from the scope and spirit of the invention. The scope of the present invention will be measured by the appended claims and their equivalents.

The invention claimed is:

1. A medical device, comprising:
a needle cannula;
a hub comprising a proximal end, a distal end and a passageway extending therethrough in fluid communication with the needle cannula, the distal end of the hub supporting the needle cannula;
a release element fixed to the hub;
a generally hollow needle shield comprising a proximal end, a distal end and a pair of wings extending laterally from opposing sides thereof, the proximal end of the needle shield removably secured to the release element with at least a grippable portion of the hub extending out from the proximal end of the needle shield, the needle shield axially movable with respect to the needle cannula from a first position in which the needle cannula is exposed to a second position in which the needle cannula is shielded within the needle shield; and
a biasing member extending between the distal end of the hub and the proximal end of the needle shield,
wherein activation of the release element releases the proximal end of the needle shield from securement, thereby causing the biasing element to axially urge the needle shield from the first position toward the second position.

2. A medical device as in claim 1, wherein the biasing member is a spring.

3. A medical device as in claim 1, wherein the outer surface of the hub and the inner surface of the needle shield interact such that axial rotation of the hub with respect to the needle shield is inhibited during movement of the needle shield from the first position toward the second position.

4. A medical device as in claim 3, wherein the hub and the needle shield have non-circular cross-sectional shapes, thereby inhibiting axial rotation of the hub with respect to the needle shield during movement of the needle shield from the first position toward the second position.

5. A medical device as in claim 3, wherein the inner surface of the needle shield includes splines for inhibiting axial rotation of the hub with respect to the needle shield during movement of the needle shield from the first position toward the second position.

6. A medical device as in claim 1, further comprising a flexible tube extending from the proximal end of the hub.

7. A medical device as in claim 1, further comprising a locking element for securing the needle shield in the second position.

8. A medical device as in claim 7, wherein the locking element comprises a pair of flexible locking arms at the distal end of the hub, each of the locking arms including a hook for releasable engagement with a corresponding detent in the proximal end of the needle shield.

9. A medical device as in claim 1, wherein the release element comprises at least one flexible locking arm extending distally from the hub and including a hook for releasable engagement with a corresponding detent in the proximal end of the needle shield.

10. A medical device as in claim 9, wherein the release element comprises a pair of flexible locking arms extending distally from the hub, each of the locking arms including a hook for releasable engagement with a corresponding detent in the proximal end of the needle shield.

11. A medical device as in claim 9, wherein the at least one flexible locking arm extends distally from the hub to form a pocket between the flexible locking arm and an outer surface of the hub adjacent the distal end of the hub, and wherein the pocket accommodates the biasing member.

12. A medical device as in claim 9, further comprising a locking element for securing the needle shield in the second position, the locking element comprising at least one flexible locking arm at the distal end of the hub and including a hook for releasable engagement with the detent in the proximal end of the needle shield when the needle shield is in the second position.

13. A medical device as in claim 1, wherein the release element comprises a rotational locking engagement between the distal end of the hub and the proximal end of the needle shield.

14. A medical device as in claim 13, further comprising a rotatable collar extending from the distal end of the hub and independently rotatable with respect to the hub, the rotatable collar in releasable engagement with the proximal end of the needle shield when the needle shield is in the first position.

15. A medical device as in claim 13, wherein the rotational locking engagement comprises a threaded engagement.

16. A medical device as in claim 1, wherein the release element comprises at least one pivot arm extending laterally along the hub, the pivot arm in releasable engagement with the proximal end of the needle shield.

17. A medical device as in claim 16, wherein the release element comprises a pair of pivot arms extending laterally along opposing outer sides of the hub, the pair of pivot arms in releasable engagement with corresponding surfaces on the proximal end of the needle shield.

18. A medical device, comprising:
a needle cannula;
a hub comprising a proximal end, a distal end and a passageway extending therethrough in fluid communication with the needle cannula, the distal end of the hub supporting the needle cannula;
a generally hollow needle shield comprising a proximal end, a distal end and a pair of wings extending laterally from opposing sides thereof, the needle shield axially movable with respect to the needle cannula from a first position in which the needle cannula is exposed to a second position in which the needle cannula is shielded within the needle shield; and
a release element fixed to the hub and removably retaining the proximal end of the needle shield with at least a portion of the hub extending from the proximal end of the needle shield, the release element comprising a rotational locking engagement the hub and the proximal end of the needle shield,
wherein activation of the release element releases the proximal end of the needle shield for movement of the needle shield from the first position toward the second position.

19. A medical device as in claim 18, wherein the rotational locking engagement comprises a threaded engagement.

20. A medical device as in claim 18, wherein the release element comprises a rotatable collar extending from the end of the hub and independently rotatable with respect to the hub, the rotatable collar in releasable engagement with the proximal end of the needle shield when the needle shield is in the first position.

21. A medical device as in claim 18, further comprising a biasing member extending between the distal end of the hub and the proximal end of the needle shield, for automatically urging the needle shield from the first position toward the second position upon activation of the release element.

22. A medical device, comprising:
a needle cannula;
a hub comprising a proximal end, a distal end and a passageway extending therethrough in fluid communication with the needle cannula, the distal end of the hub supporting the needle cannula;
a generally hollow needle shield comprising a proximal end, a distal end and a pair of wings extending laterally from opposing sides thereof, the needle shield encompassing the hub with at least a portion of the hub extending from the proximal end of the needle shield, the needle shield axially movable with respect to the needle cannula from a first position in which the needle cannula is exposed to a second position in which the needle cannula is shielded within the needle shield; and
a biasing member extending between the hub and the needle shield and exerting a biasing force to urge the needle shield distally toward the second position,
wherein when the needle shield is in the first position, the shield and the hub exhibit a mutual friction greater than the biasing force exerted by the biasing member when the needle shield is in the first position.

23. A medical device as in claim 22, wherein the mutual friction between the shield and the hub is about 120% to about 1000% of the biasing force exerted by the biasing element.

24. A medical device as in claim 22, wherein the mutual friction is provided through surface texture on at least one of an outside surface of the hub or an inside surface of the needle shield.

25. A medical device as in claim 22, wherein the portion of the hub which extends from the proximal end of the needle shield includes a surface grip and the needle shield includes a surface grip, such that opposing forces applied against the surface grip of the hub and the surface grip of the needle shield releases the mutual friction between the hub and the shield, and the biasing element causes axial movement of the needle shield toward the second position.

26. A medical device as in claim 22, further comprising a locking element for securing the needle shield in the second position.

27. A medical device as in claim 26, wherein an inner surface of the needle shield includes a first surface feature at the proximal end thereof and a second surface feature at the distal end thereof and the external surface of the hub includes a surface feature for interference engagement with the first surface feature of the needle shield when the needle shield is in the first position for retaining the needle shield in the first position, and for interference engagement with the second surface feature of the needle shield when the needle shield is the second position for securing the needle shield in the second position.

28. A medical device as in claim 26, wherein the first and second surface features of the needle shield comprise first and second detents, respectively, and the surface feature of the hub comprises a bump for respective interference engagement with the first and second detents of the needle shield.
